(19) United States
(12) Patent Application Publication
(10) Pub. No.: US 2004/0022734 A1
(43) Pub. Date: Feb. 5, 2004

(54) Dihydroporphyrin derivatives and their uses

(76) Inventors: Ludovic Bourre, Nannes sur Loire (FR); Thierry Patrice, Saint Herblain (FR)

Correspondence Address: YOUNG & THOMPSON, 745 SOUTH 23RD STREET 2ND FLOOR, ARLINGTON, VA, 22202

(21) Appl. No.: 10/343,679

(22) PCT Filed: Jul. 27, 2001
(86) PCT No.: PCT/FR01/02470

(30) Foreign Application Priority Data
Aug. 1, 2000 (FR) 00/10165

(51) Int. Cl. A61K 31/675; C07D 487/22; A61K 31/555; H01L 31/00; A61K 31/409

(52) U.S. Cl. 424/9.61; 540/145; 514/80; 514/185; 514/410; 136/263

(57) ABSTRACT

The invention relates to a compound derived from dihydroporphyrin having the general formula (I):

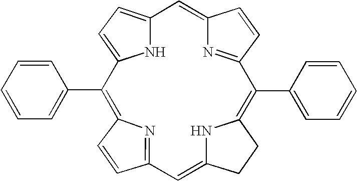

in which the phenyl groups are substituted or not; or one of its salts, or one of its metallic complexes.

Application: treatment by photochemotherapy.

The invention relates to new compounds derived from dihydroporphyrin and their applications, particularly in the fields of photochemotherapy, in particular for the treatment of cancer, of fluorescent imaging and optoelectronics.

The porphyrins and their derivatives have been the object of numerous studies in recent years because of their wide field of potential application. Thus, U.S. Pat. Nos. 5,162,519, 4,992,257, 4,837,221, 5,162,519 and 5,703,230 show such examples of compounds.

Among these numerous fields of application, photochemotherapy seems now to be one of the most promising fields. Photochemotherapy is a technique of treatment which has been developed for several years, in particular for the treatment of cancer. This technique comprises the administration of a photosensitive agent, of low toxicity, which will be retained with relative selectively by the tissues of high mitotic index, in particular neoplastic tissues. This photosensitive agent is thus excited by luminous radiation of a wavelength adapted to the luminous absorption spectrum of the photosensitizer. The luminous radiation absorbed by the photosensitive molecule gives rise to type 1 reactions (production of hydroxyl radicals) or type 2 reactions (production of atomic oxygen) by an intersystem conversion mechanism. These radicular species give rise to oxidation and peroxidation reactions in the tissues which have fixed the photosensitizer and then cellular death.

The effectiveness of the technique relies on the predetermined properties of the photosensitive agent. Thus, to give rise to the desired phototoxic effect at a depth within a tissue of a substance, it is necessary to use photosensitizers which have high coefficients of absorption at wavelengths greater than 650 nm, at which the bodily tissues are the most transparent to light (see Sternberg et al., “An Overview of Second Generation Drugs for Photodynamic Therapy including BPD-MA (Benzoporphyrin Derivative) Photodynamic Therapy and Biomedical Lasers, 470-4 (Spinelli et al. eds. 1992).

Similarly, the photosensitive agent must on the one hand have a rapid speed of penetration into the target cells to be destroyed so as to reduce the time necessary between administration of the photosensitive agent and the luminous radiation, on the other hand a short retention time in the healthy regions to reduce the secondary effects and the time of hospitalization as well as the risk of iatrogenic burning during untimely exposure to light. There is thus continuously the search for new photosensitive molecules having a shorter retention time in the target regions of the organism of a living creature, this shorter lifetime being of interest in other fields of application, in particular medical imaging and optoelectronics.

It is to satisfy these requirements for rapid capture by the target tissues and rapid elimination by the other tissues, in comparison to existing compounds, that the molecule according to the present invention has been synthesized.

To this end, the invention has for its object a new compound of dihydroporphyrin having the general formula (I):

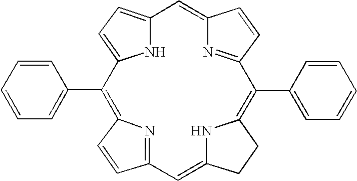

in which the phenyl groups are substituted or not; or one of its salts, or one of its metallic complexes.

The compounds of the invention can exist as mentioned above in the acid or basic addition salt condition or in the metallic complex condition.

The invention also has for its object a compound derived from dihydroporphyrin of the mentioned type having the general formula (II):

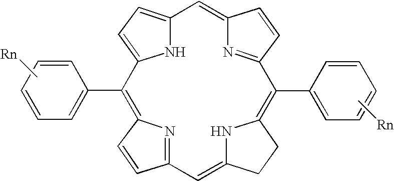

in which the phenyl group is substituted, n is equal to 1 to 5, preferably equal to 1 to 3, and each R substituent, which can be identical or different, and in identical or different positions on its substituted phenyl group, is hydroxy (—OH), amino (—NH₂), sulphydril (—SH), phosphonate (PO₃H₂, PO₃Na₂), ethylphosphonate (PO₃Et₂), sulfonate, aromatic, alkyl substituted or not, cycloalkyl substituted or not, aliphatic, amino acid, peptide or polypeptide, pyridine with different positions for the nitrogen atom, purine, pyrimidine, nucleoside, saccharide, polysaccharide, carboxylic acid, an amide group, an ester group, a quaternary ammonium substituted or not.

By aliphatic above, there is meant in particular one or several amino acids that are not cyclic, namely, for example, serine or a polyethylene glycol chain (PEG) or any other substituent.

By aromatic group mentioned above, there is meant in particular one or several amino acids comprising at least one aromatic cycle, for example Phe or Tyr or any other aromatic group carbonated or not.

Again, the compounds described above can be in the form of derivatives such as acid or basic addition salts, metallic complexes, for example Zn, Ga, Pa, or hydrates or other solvates, in particular with lower aliphatic alcohols Of C₁-C₆.

The invention will be better understood from a reading of the following description with examples of embodiment, with reference to the accompanying drawings, in which:

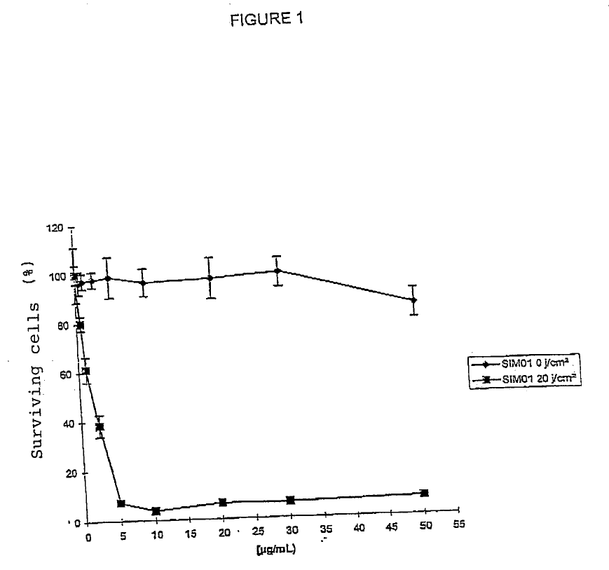

FIG. 1 represents the toxicity and phototoxicity of SIMO1 cells on C6 cells incubated for 5 hours;

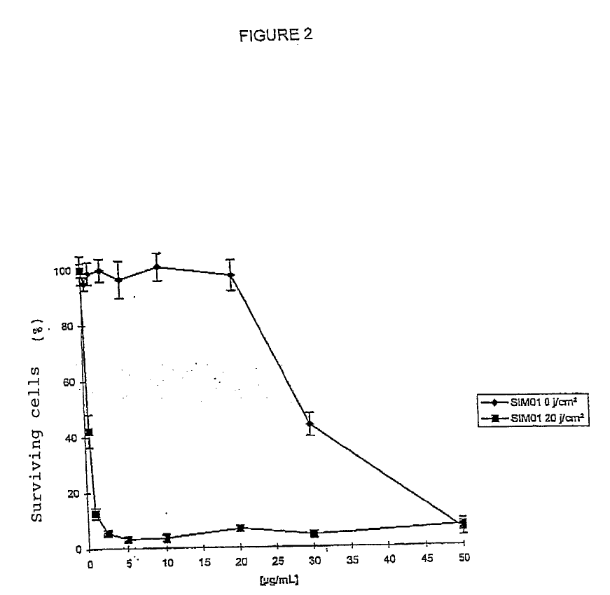

FIG. 2 represents the toxicity and phototoxicity of SIMO1 cells on C6 cells incubated for 20 hours;

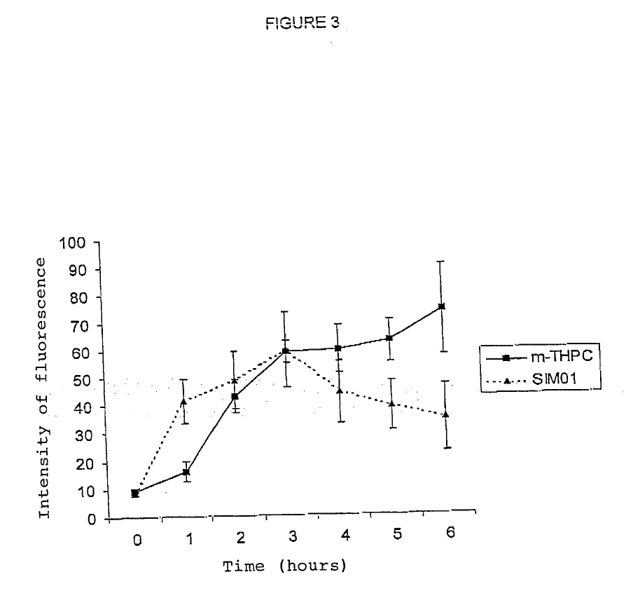

FIG. 3 represents, in the form of curves, the intensity of intracellular fluorescence of the SIMO1 molecules and m-THPC;

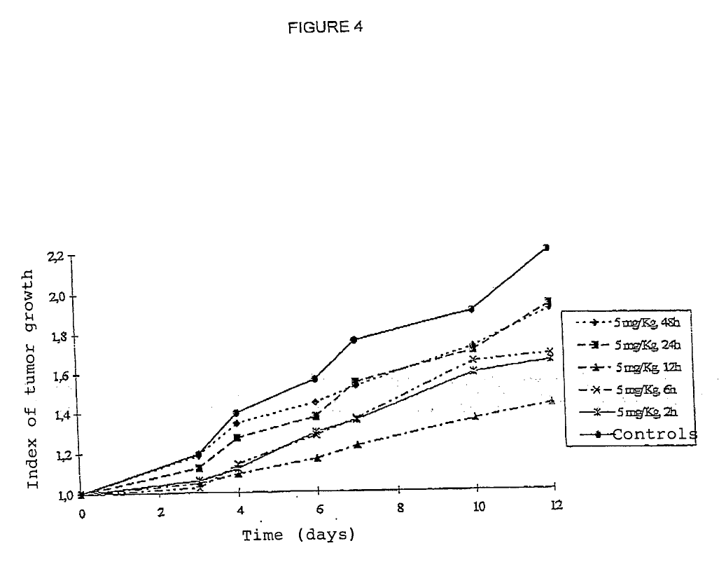

FIG. 4 represents, in the form of curves, the increase of a tumor measured at different intervals of time after injection with SIMO1;

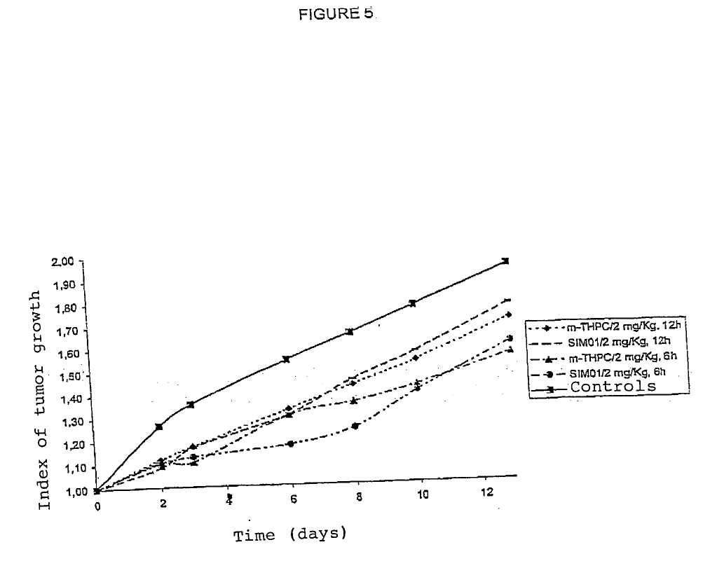

FIG. 5 represents curves analogous to FIG. 4 after injection of SIMO1 or m-THPC and

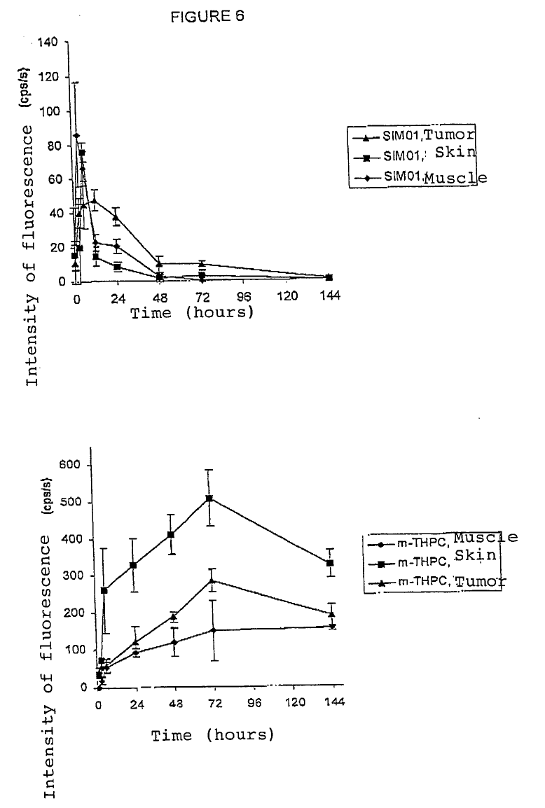

FIG. 6 represents curves of spectrofluorometric measurement on different tissues of mice grafted at different intervals of time after injection of m-THPC or SIMO1.

The example which follows shows the preparation of a preferred compound of the invention.

The synthesis of this compound, corresponding to the general formula (III),

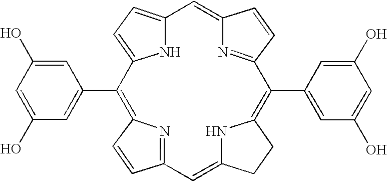

and which will be called hereafter for simplicity SIMO1, comprises the following steps.

Step 1: dipyrrylmethane

Dipyrrylmethane has been prepared as mentioned above in Wang and Bruce, Synlett, 1267, 1995.

Step 2: 5,15-Bis(3,5-dimethoxy-1-phenyl)porphyrin

Di-methoxy-3,5-benzaldehyde (341 mg, 2.05 mmole), dipyrrylmethane (300 mg, 2.05 mmole) were added to 205 ml of dichloromethane distilled in a flask with a circular bottom. The solution was subjected to ultrasound in a nitrogen flow for 20 minutes. Then the trifluoroacetic acid (47 ml, 615 mmole) was injected and the mixture was agitated at ambient temperature overnight.

A solution of DDQ 2,3-Dichloro-5,6-dicyano-1,4-benzoquinone (0.931 g in 10 ml of toluene) was added and the reaction mixture was refluxed for 30 minutes with an oil bath preheated to 60° C.

The purification of the porphyrin was achieved by flash chromatography eluted with hexane/CH₂Cl₂: 5/5 and yielded 253 mg of the desired porphyrin (36%).

RMN 1H (CF3COOD) ppm: 11:13 (H meso), 9.7 and 9.3 (2H, d, J=6 Hz, pyrrole), 7.9 (o-H, phenyl), 7.4 (p-phenyl), 4.2 (methoxy) UV-Vis (CH₂Cl₂), max (nm): 407, 502, 536, 574, 629. FAB-MS: calculated for C36H30N404: 582.2 found: 583 (M+H+).

Step 3: 5,15-Bis(3,5-dihydroxy-1-phenyl)porphyrin

The compound of step 2 (90 mg, 0.15 mmole) was dissolved in dry CH₂Cl₂ (10 cm³) at −20°, followed by the addition of BBr₃ (0.12 cm³, 1.24 mmole). The resulting green solution was agitated for 12 hours and then placed ice water. Methanol (20 cm³) and then ethyl acetate (10 cm³) were added and the suspension and the mixture was neutralized with NaHCO₃. The organic layer was separated, washed first with a solution of NH₄Cl then twice with water and dried on anhydrous sodium sulfate. The resulting solution was evaporated on a rotating evaporator and the residue re-dissolved in acetone. After addition of pentane, a precipitate provided the compound of step 3 with a yield of 52%. RMN 1H (acetone d6): 10.55 (H meso), 9.6 and 9.3 (2H, d, J=6 Hz, pyrrole), 7.3 (o-H, phenyl), 6.9 (p-phenyl), −3.1 (NH). UV-Vis (acetone), max (nm): 402, 532, 572, 628.

Step 4: 2,3-dihydro-5, 15-Bis(3,5-dihydroxy-1-phenyl)-prohyrin (SIMO1)

The compound of step 3 (42 mg, 0.08 mmole), anhydrous K₂CO₃ (0.32 g) and anhydrous pyridine (5 cm³) were heated (120° C.) for 5 minutes. Then, small quantities (0.25 cm³) of a mixture of p-toluenesulfonylhydrazide (0.44 g) in pyridine (2.5 cm³) were added each 15 minutes for 2.5 hours. After cooling, the solution of pyridine was evaporated under vacuum. The resulting powder was re-dissolved in a mixture of 1/1 acetone/ethyl acetate, then washed twice with water and then saturated with a bicarbonate solution. O-chloranil was added in portions to the organic solution at ambient temperature until the absorption peak of 735 nm disappeared. The solution was washed twice with NaHSO₄ (5%), distilled water, NaOH (0.1 M) and saturated bicarbonate. After drying on MsSO₄, the solvent was evaporated in a rotating evaporator. The residue was crystallized from acetone/pentane to provide 30 mg of the compound of step 4 (yield 74%) which will be called hereinafter SIMO1.

RMN 1H (acetone, d6) ppm: 10.1 and 9.2 (2H-meso), 9.35-8.6 (6H, m, pyrrole), 7.2 and 6.95 (4H, d, o-phenyl), 6.85 and 6.75 (2H, t, p-phenyl), 4.65 and 4.45 (4H, m, pyrrolidine), −1.4 and −1.9 (2H, s, NH). UV-Vis (acetone), max (nm): 395, 405, 500, 645.

The method of preparation of these compounds is different from that used for m-THPC (U.S. Pat. No. 5,162,519). It implies a reaction between an aromatic aldehyde and dipyrrylmethane, which leads to the first step of the synthesis of SIMO1 whilst m-THPC involves the reaction of pyrrole and aromatic aldehydes.

The compounds of the invention were the objects of tests which demonstrated their therapeutic properties.

To this end, the compounds of the invention can be used in the form of medications characterized in that they consist in one of the mentioned compounds or in the form of a pharmaceutical composition containing a compound of the mentioned type, associated with an excipient.

The compounds of the invention can be present in any form of composition administrable to the human or animal body and suitable for enteral, parenteral or transdermal administration, such as tablets, dragees, skin cream, gels, capsules, suspensions or ingestible or injectable solutions, such as syrup or ampoules, transdermal patches, liposomal formulations (nanoparticles) etc. associated with suitable excipients and in dosages to permit one or several cures comprising one or several daily administrations for one or several days at a dosage level comprised for example between 0.1 and 20 mg/kg of weight.

The preferred administrable forms are injectable, in particular in the form of an intramuscular or intravenous injection or in the form of a transcutaneous application.

The tests described below relate to the activity of the SIMO1 molecule. The activity of this new SIMO1 molecule has been tested in vitro with tests of toxicity and phototoxicity and in vivo by a study of phototoxicity by evaluation of the retardation of the growth of tumors relative to untreated animals.

The pharmaco kinetics of SIMO1 have also been studied to determine the retardation for a maximum incorporation of SIMO1 compared with the retardation by incorporation of the m-THPC (conventional molecule widely used in the field of photo-chemotherapy). The analysis of the elimination of SIMO1 relative to m-THPC has also been studied.

SIMO1 is a porphyrin derivative of molecular weight 524. SIMO1 shows, in a saline isotonic solution, 3 principal absorption spectra at 428, 513 and 652 mm. The saline solution was obtained as follows: one milligram of SIMO1 was dissolved in 1 ml of solvent (50% water, 30% PEG and 20% pure ethanol at 99%). Other concentrations were obtained successively by addition of a saline isotonic solution.

To study the toxicity and phototoxicity of the SIMO1 molecule, the following procedure was carried out:

C6 cells were cultured in flasks of 25 cm² (Polylabo, Strasbourg, France) in an RPMI medium supplemented with 10% (V/V) of fetal calf serum (FCS), 100 units of penicillin mL⁻¹, 100 mg of streptomycin and 2 mM of glutamine.

The cells were replicated by dispersion culturing with 0.025% of trypsin in 0.02% ethylene-diaminotetracetic acid (EDTA) for a contact time of 2 minutes and returned to adhere at a dilution of 1:3, which maintain the cells in the exponential phase of growth. The cells were verified on a regular basis by contamination of the mycoplasms.

The cells thus cultivated were subjected to a photodynamic treatment. Aliquots (11 μl) of the photosensitizing solution SIMO1 were added to C6 cells adhered in wells of plates with 96 wells after trypsonation, as described above. The cellular concentration was 5.10⁴ cells/ml⁻¹ (100 μl per well). The final concentration of the solutions of SIMO1 was comprised within the range of 0.5 to 50 μg/ml. Immediately after addition of the photosensitizer SIMO1, the plates of cells were held in complete darkness till the time of counting, except for a laser irradiation of the cells treated with PDT. The fresh medium containing FCS but free from photosensitizer was prepared before laser irradiation. A laser with a diode at 650 nm was used. The power of the end of the fiber was adjusted, by using a device for measuring power (Coherent, France), at 500 mW. The light was transmitted to the target by an optical fiber at a distance of 20 mm so as to irradiate the cells in the wells 6 mm in diameter within a single field providing an illumination of all of the region. The time of exposure was 13 seconds per well at 650 nm, supplying an energy density of 20 J/cm².

The cells were incubated with SIMO1 for 5 or 20 hours. For phototoxicity tests, the cells were washed and the medium was replaced immediately before laser irradiation.

Cell counts were carried out 24 hours after the end of the experiments so as to avoid low estimations of the surviving cells by spacing the altered but living cells. At the time of counting, 15 μl of saline buffer solution with phosphate (PBS-MTT solution, 5 mg/ml⁻¹) were added to the wells. After 4 hours, 150 μl of isopropanol-HCl 0.04N was added according to the method described by Mosmann (T. Mosmann. Rapid calorimetric assay for cellular growth and survival: application to proliferation and cytotoxicity asay. J. Immunol. Methods, 65, 55-63, 1985).

The optical density of each well of the microplates was then read at 570 nm by means of a Uniskan Titertack (Flow Laboratories, Puteaux, France). An apparatus containing RPMI without phenyl red and with 15 μl of an MTT solution was used to determine the blanks for the measurements of absorbents.

The results are given in FIGS. 1 and 2, in which FIG. 1 represents the toxicity and phototoxicity of SIMO1 on C6 cells incubated for 5 hours with SIMO1 non-irradiated or irradiated with a diode laser at 652 nm at 20 joules/cm² then treated with MTT.

FIG. 2 is distinguished from FIG. 1 by the time of incubation of the C6 cells with SIMO1, fixed at 20 hours.

As will be seen in these figures, the SIMO1 molecule shows great activity in vitro with good effectiveness at low concentrations. For phototoxic treatment (20 joules/cm²), after an incubation time of 5 hours, the DL₅₀ was 1.75 μg/ml and after 20 hours of incubation 0.45 μg/ml. As to SIMO1 toxicity, no toxicity was detected after 5 hours of incubation and after 20 hours of incubation, the DL₅₀ was 29 μg/ml. These tests clearly show the harmlessness of SIMO1 in the absence of luminous irradiation short of a concentration of about 20 μg/ml and, conversely, its certain phototoxicity upon irradiation.

So as to confirm the potential use of the mentioned compounds for the production of a photosensitive composition administrable to a living creature and useful as an agent adapted to give rise, under the influence of irradiation, to cytolysis or necrosis at least partially of at least one target region of the human or animal body, a visualization was conducted of the localization in vitro of the SIMO1 molecule, as well as the determination of the pharmacokinetics compared to m-THPC, by fluorescent imaging so as to confirm that the mentioned compounds were capable of penetrating the cells. Thus, C6 cells were seeded at a rate of 10⁵ cells/ml on circular glass plates after trypsinization. After 24 hours, cells were incubated with SIMO1 (10 μg/ml) at 37° C. for 3 hours and then washed with PBS (pH 7.2). Fluorescence analysis of SIMO1 (emitting at 650 nm) was carried out after excitation of 450 nm to 480 nm with a 150 watt Xenon lamp by using a black and white video camera with very sensitive detection of photons (Kappa CF 8/4; Fischer Scientific S.A., France), connected to an optical microscope (Olympus BX 40, France) provided with an oil immersion objective magnifying 100 times.

The strongest intensity of fluorescence was located principally in the cytoplasm of the C6 cells. The images produced immediately after incubation for 3 hours with SIMO1 showed clearly that the compounds are capable of penetrating the cells.

As to pharmacokinetics (FIG. 3), the maximum concentration of SIMO1 was observed 3 hours after incubation, with a high incorporation as early as one hour, then a decrease in the intensity of fluorescence was observed from 3 hours to 6 hours. For m-THPC, the intracellular fluorescence increases to 6 hours, with a small incorporation of photosensitizer 1 hour after incubation. The incubation as well as the more rapid elimination of SIMO1 is due to the more hydrophilic character of the molecule, which has only two phenyls about the central tetrapyrol nucleus compared to m-THPC which has four of them.

The in vitro tests were followed by toxic and phytotoxic tests in vivo so as to demonstrate the interest of the mentioned compounds particularly in the form of compositions useful for diagnostic or treatment purposes by demonstrating that these compositions, which comprise at least one photosensitive agent, are adapted to induce in vivo when they are subjected to luminous irradiation and a predetermined wavelength, a necrosis or a cytolysis at least partially of at least one target zone of the human or animal body, this necrosis or cytolysis being measured particularly in terms of retardation of growth of a tumor relative to a reference tumor. These tests have permitted determining on the one hand the optimum period of time that should be left to elapse between injection and the laser irradiation, on the other hand the effectiveness of the SIMO1 molecule by comparison with known molecules, especially m-THPC which is a chlorine derivative photosensitive agent.

To establish these conclusions, the following procedure was used: Swiss male nude/nude mice aged 7 to 9 weeks (weight range 25-35 g) were obtained from Iffa-Credo (L'arbresle, France).

The test method is that of the cellular tumor HT29 obtained initially from human colorectal adenocarcinoma.

The tumor grafts were obtained as follows: nonnecrotic solid tumor tissues (diameter 1 to 2 cm) were removed immediately after death of the donor mouse and then mechanically crushed in 1 ml of a 0.9% saline solution. This solution (0.2 ml) was injected sub-cutaneously into the rear paw of each mouse. This tumor was used one week later, when its diameter was 18-20 mm. SIMO1 was injected intra-perintineally in a saline solution and 12 hours later the mouse was anesthetized and the tumor exposed to light of 300 J/cm⁻². The wavelength was illumination was selected between 652-653 nm.

The light source was a diode laser at 652 nm.

The luminous density was maintained below 0.3 W.cm⁻², at doses at which the thermal effects are indetectable. A single irradiation was carried out and the time of exposure was calculated as a function of the diameter of the tumor to obtain an energy density of 300 J/cm⁻² at a predetermined constant light density.

Each day, the index of tumor growth was measured by using a slide foot, the principal orthogonal diameters being added and divided by a factor of 2.

A statistical characteristic of the growth index between treated and control mice (without light or SIMO1) was carried by using the Student test.

All the tested treatment conditions comprising the photosensitive agent and the light were seen to produce a reduction of growth of the tumor compared to the untreated control group. The most marked effects were observed for a treatment including a retardation of 12 hours between intra-peritoneal injection and laser irradiation. Twelve days after treatment, the growth of the tumor had slowed by 40%. These results are illustrated in FIG. 4, in which the growth of a tumor induced by HT-29 is measured after irradiation at 300 J/cm² with a diode laser 652 nm for 2 hours, 6 hours, 12 hours, 24 hours and 48 hours after intra-peritoneal injection of 5 mg/kg of SIMO1. A control group having received no exposure to light or photosensitive agent was constituted.

FIG. 5 shows the same experiments as FIG. 4 carried out for groups of mice treated either with SIMO1, or with m-THPC and subjected to irradiation 6 hours and 12 hours after intra-peritoneal injection of 2 mg/kg of SIMO1 or of m-THPC. There will be noted a comparable activity between the two molecules.

New tests were carried out to determine the period at which is noted a maximum incorporation of the compounds in the tissues as a function of the nature of the tissues and of the time of retention of the compound by said tissues. These tests were carried out in a comparable manner for SIMO1 and m-THPC.

For these tests, the following was the procedure: spectrofluorimetric measurements were carried out on mice grafted with HT-29 at different intervals of time after injection of 2 mg/kg⁻¹ of m-THPC or SIMO1. The tested times were 3, 6, 12, 24, 48 and 144 hours on mice. The results are given in FIG. 6.

The level of fluorescence were recorded in different tissues, namely the muscle, the skin and the tumor. The measurements were taken by using an optical fiber placed directly in contact with the tissues. At least four spectra per tissue and per mouse were recorded (at least five mice were used for each experimental condition). Muscle, skin and tumor were successively studied.

The peak of fluorescence was observed at 650 nm for SIMO1 and m-THPC. The emission spectra of fluorescence of untreated mice tissue were recorded at three separate times so as to estimate the intermouse variations. The values of fluorescent intensity mentioned in FIG. 6 correspond for each organ to the result of the difference between the value of fluorescence obtained in a treated mouse and the value of fluorescence recorded in the control group. The results of spectrofluorometric measurement in vivo were expressed in beats per second (arbitrary unit).

The results are given in FIG. 6. The SIMO1 molecule shows retention time shorter in all the tissues, compared to the m-THPC molecule. The maximum incorporation for SIMO1 is noted between 6 and 12 hours after injection, whilst the maximum for m-THPC is reached between 48 and 72 hours. For SIMO1, no photosensitive agent was detected after 48 hours, whilst for m-THPC, the photosensitive agent was still detectable 144 hours after injection. The SIMO1 molecule thus shows clearly a time of incorporation and a time of retention shorter than the photosensitive agents known until now. This shorter retention time permits reducing the time of hospitalization of the patients, limiting the secondary effects of such severe treatments in these latter, and avoiding treatments against luminous irradiation because the medication is rapidly eliminated from the organism.

These kinetics of more rapid incorporation associated with more rapid elimination, relative to m-THPC or other compounds, must be connected to the original structure of the molecule according to the invention, which has only two phenyls.

The specific properties of the compounds mentioned above in terms of time of retention and time of incorporation in the tissues to be treated, permit foretelling interest in said compounds in other applications, in particular as markers for medical imaging devices, these markers being characterized in that they contain as fluorescent marking agent at least one of the mentioned compounds.

The mentioned compounds could also be retained in the scope of application of the production of photocells, at least one of the constituent layers of the photocell being a compound of the mentioned type.

Obviously, as was mentioned above, a composition incorporating such compounds could, in the case of application to therapeutic purposes or diagnostics, be present in various forms, in particular in the form of tablets, pills, capsules, granules and powders for dissolution, etc. administrable orally or enterally. They could also be present in the form of a injectable preparation for parenteral administration or else in the form of suppositories. They could also be prepared in the form of a topical preparation present in the form of a cream, a powder, a liquid or the like locally applicable to said region to be treated.

The doses of this photosensitive agent will depend on the symptoms, the age and the sex of the patient and possibly on other factors, in particular the type of photosensitive agent used in the scope of the treatment by photochemotherapy and the source of the light used.

In conclusion, these compounds, which clearly suit the present needs in the field of photochemotherapy, should in the future find other applications, particularly in the field of medical imaging and that of opto-electronics.

1. Compound derived from dihydroporphyrin having the general formula (I): 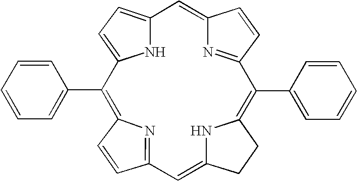
in which the phenyl groups are substituted or not; or one of its salts, or one of its metallic complexes.

2. Compound derived from dihydroporphyrin according to claim 1 having the general formula (II): 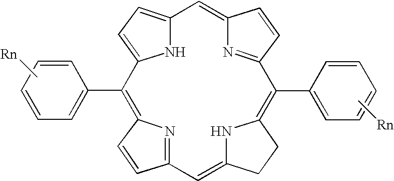
in which the phenyl group is substituted, n is equal to 1 to 5, preferably equal to 1 to 3, and each R substituent, which can be identical or different, and in identical or different positions on its substituted phenyl group, is a hydroxy group (—OH), amino (—NH₂), sulphydril (—SH), phosphonate (PO₃H₂, PO₃Na₂), ethylphosphonate (PO₃Et₂), sulfonate, aromatic, alkyl substituted or not, cycloalkyl substituted or not, aliphatic, amino acid, peptide or polypeptide, pyridine with different positions for the nitrogen atom, purine, pyrimidine, nucleoside, saccharide, polysaccharide, carboxylic acid, an amide group, an ester group, a quaternary ammonium substituted or not.

3. Compound derived from dihydroporphyrin according to one of claims 1 and 2,
characterized in that it has the general formula (III):

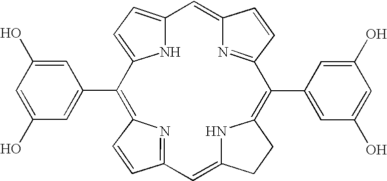

4. Medication,
characterized in that it consists in a compound according to one of claims 1 to 3.

5. Pharmaceutical composition,
characterized in that it contains a compound according to one of claims 1 to 3 in combination with an excipient.

6. Composition useful for diagnostic or treatment purposes of the type comprising at least one photosensitive agent adapted to induce in vitro or in vivo when it is subjected to luminous irradiation at a predetermined wavelength, necrosis or cytolysis at least partially of at least one target zone of the human or animal body,
characterized in that said photosensitive agent is a compound according to one of claims 1 to 3.

7. Composition according to claim 6,
characterized in that it is present in the form administrable to the human or animal body by digestive or parenteral route, in particular by intra-muscular or intravenous injection or by transcutaneous application.

8. Composition according to one of claims 6 and 7,
characterized in that it is administered at a dose comprised within the range 0.1 mg/kg-20 mg/kg.

9. Marker for a medical imaging device,
characterized in that it contains as a fluorescent marking agent at least one compound according to one of claims 1 to 3.

10. Photocell,
characterized in that at least one of the constituent layers of the photocell is a compound according to one of claims 1 to 3.

11. The use of compounds according to one of claims 1 to 3 for the production of a photosensitive composition administrable to a living being and useful as an agent to give rise, under the influence of luminous irradiation, to cytolysis or necrosis at least partial of at least one target zone of the human or animal body.
